(19) United States
(12) Patent Application Publication
(10) Pub. No.: US 2025/0057426 A1
(43) Pub. Date: Feb. 20, 2025

(54) COMPUTER-IMPLEMENTED METHODS AND ASSOCIATED SYSTEMS FOR DETECTING MALIGNANCY

(71) Applicant: The Trustees of the University of Pennsylvania, Philadelphia, PA (US)

(72) Inventors: Sunil Singhal, Pennington, NJ (US); Feredun Azari, Philadelphia, PA (US); Sardar Azari, Vienna, VA (US)

(21) Appl. No.: 18/720,823

(22) PCT Filed: Dec. 15, 2022
(86) PCT No.: PCT/US22/81707

§ 371 Date: Jun. 17, 2024

Related U.S. Application Data
(60) Provisional application No. 63/291,179, filed on Dec. 17, 2021.

(51) Int. Cl. A61B 5/00

(52) U.S. Cl. CPC A61B 5/0071; A61B 5/004; A61B 5/7267; A61B 5/7275; A61B 2505/05; A61B 2576/02

(57) ABSTRACT

A computer-implemented method is provided. The computer-implemented method includes: receiving a fluorescence image of a patient tissue; determining a level of fluorescence for each of a plurality of sections of the fluorescence image; comparing a level of fluorescence for a section of the fluorescence image to one or more other levels of fluorescence for one or more other sections of the fluorescence image; determining a TBR value for the level of fluorescence for the section of the fluorescence image based on the comparing, wherein the comparing step is according to the TBR value; repeating steps for another fluorescence image after the patient tissue is resected; receiving a plurality of medical-history parameters of the patient; and calculating whether the patient tissue contains a malignant tumor from TBR values of a plurality of sections of the fluorescence image and the plurality of medical-history parameters.

### CROSS-REFERENCE TO RELATED APPLICATION

The present application claims priority to U.S. Provisional Patent Application No. 63/291,179, filed Dec. 17, 2021, which is incorporated herein by reference in its entirety.

### BACKGROUND

Tumor malignancy can be identified through various processes, including monitoring cancer biomarkers in a patient's blood, conducting a biopsy, imaging test such as X-ray, ultrasound, MRI, proteogenomics, liquid biopsies, and the like.

### SUMMARY

One aspect of the present disclosure provides a computer-implemented method including: (a) receiving a fluorescence image of a patient tissue; (b) determining a level of fluorescence for each of a plurality of sections of the fluorescence image; (c) comparing a level of fluorescence for a section of the fluorescence image to one or more other levels of fluorescence for one or more other sections of the fluorescence image; (d) determining a tumor-to-background ratio (TBR) value for the level of fluorescence for the section of the fluorescence image based on the comparing, wherein the comparing step is according to the TBR value; (e) repeating steps (a)-(d) for another fluorescence image after the patient tissue is resected; (f) receiving a plurality of medical-history parameters of the patient; and (g) calculating, via a machine-learning model previously trained using at least the TBR values and the medical-history parameters, whether the patient tissue contains a malignant tumor from TBR values of a plurality of sections of the fluorescence image and the plurality of medical-history parameters, wherein the machine-learning model is further trained using a location of the patient tissue as a parameter.

In certain aspects, the computer-implemented method, further includes: (h) repeating steps (a)-(d) for yet another fluorescence image. In certain aspects, steps (a)-(d) are performed after resection, but before removal of the patient tissue from the patient's body. In certain aspects, steps (a)-(d) are performed after resection and before removal of the patient tissue from the patient's body. In certain aspects, each of the plurality of sections include a pixel of the fluorescence image. In certain aspects, the patient tissue is an organ. In certain aspects, the organ is a lung. In certain aspects, the patient tissue contains a tumor or lesion.

In certain aspects, the plurality of medical-history parameters include: a patient age, a patient gender, a smoking history of the patient, a time period between fluorescence infusion and surgery for the patient; a distance of the patient tissue from a pleural surface, a size of a tumor contained within the patient tissue, a positron emission tomography (PET) standardized uptake value (SUV) for the fluorescence image; an American Society of Anesthesiologists (ASA) classification for the patient, and a length of stay for the patient. In certain aspects, the machine-learning model includes an Image Segmentation algorithm. In certain aspects, the computer-implemented method further includes: categorizing a subset of the plurality of sections of the fluorescence image as background sections of the patient tissue; and categorizing another subset of the plurality of sections of the fluorescence image as containing sections of tumor of the patient tissue. In certain aspects, steps (a)-(f) are performed in less than a minute. In certain aspects, the computer-implemented method further includes: inputting another fluorescence image of another patient tumor; inputting plurality of medical-history parameters for the other patient; and categorizing a subset of a plurality of sections of the other fluorescence image as containing a tumor. In certain aspects, the computer-implemented method further includes: categorizing whether the tumor is benign or malignant.

Another aspect of the present disclosure provides a smart device configured to implement any of the methods described herein. The smart device includes an optical imaging system including a camera optic configured to image at least a section of a tissue of a patient, the optical imaging system being configured and adapted to detect near infrared (NIR) fluorescence dye injected into the patient. The smart device also includes a light source. The smart device also includes a computing device configured and adapted to utilize optical imaging and quantify background and tumor fluorescence in real time based on predictive models.

In certain aspects, the computing device is configured and adapted to assess the probability of lesions being malignant. In certain aspects, the smart device includes processors and memory capable of performing TBR analysis for images captured by the optical imaging system, the TBR analysis including: delineating areas of high fluorescence; delineating areas of low fluorescence; and calculating a TBR from delineating the areas of high fluorescence and the areas of low fluorescence. In certain aspects, the computing device is capable of: detecting fluorescence, determining the TBR, and determining a probability of malignancy.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A depicts an example representation of a tumor-to-background ratio (TBR) calculation in an operating room (OR) after a wedge resection of pulmonary adenocarcinoma. Panel A depicts a near infrared (NIR) rendering of a lesion (tumor). Mean fluorescence intensity of the tumor and background parenchyma is calculated using Image J software. TBR in this example is calculated at 2.8. FIG. 1B depicts a white light image of the resected tumor. FIG. 1C depicts a NIR overlay with white light image, allowing for optimal real time nodule detection intra-operatively.

FIG. 2 depicts a receiver operating characteristic (ROC) graph, with area under the curve (AUC) at 0.897, for a malignancy detection algorithm according to an embodiment of the present disclosure.

FIGS. 3 and 4 depict predicted odds ratios for selected parameters of a malignancy detection algorithm according to embodiments of the present disclosure.

FIG. 5 depicts a malignancy probability display based on intra-operative findings according to an embodiment of the present disclosure.

FIGS. 6A-6D depict image processing for a patient tissue according to an embodiment of the present disclosure. FIG. 6A depicts an original NIR image of a patient tissue. FIG. 6B depicts a tumor fluorescence calculation rendering of the patient tissue. FIG. 6C depicts a tumor background selection rendering for the patient tissue. FIG. 6D depicts a rendering of a TBR calculation for the patient tissue.

FIGS. 7A-7C depict an example representation of in-situ TBR calculation in the operating room after a wedge resection of pulmonary adenocarcinoma. FIG. 7A represents a near-infra-red (NIR) rendering of the lesion (tumor). Mean fluorescence intensity of the tumor and background parenchyma can be calculated using image J software. Ratio is of tumor to background is calculated to be 8.9. FIG. 7B represents a NIR overlay with white light image, allowing for optimal real time nodule detection intra-operatively. FIG. 7C represents a white light image and panel.

FIGS. 8A-8C depict association of in situ TBR measurements with various patient factors including age, gender, smoking history, and time from infusion to surgery for primary lung adenocarcinomas. FIG. 8D depicts scatter plots show a lack of correlation with significantly small R² value. Line of interpolation looking at in situ TBR vs age shows no correlation. FIGS. 8E-8H depict similar results as obtained for binned box plots.

FIGS. 9A-9C depict depict in situ TBR measurement variation with depth of the lesion, pet SUV by gender, SUV, and tumor size are presented in scatter plot with associated linear regression and calculated coefficient of determination (R2). Lesions deeper than 1.0 cm were likely to have lower in situ TBR, meaning less observed fluorescence, compared to lesions on the pleural surface (p<0.01). FIG. 9D 9G depict subgroup analyses of correlation of lesions size, location, PET SUV, and depth presented in a box plot format. Circles represent values that are outliers that were not included in the statistical analysis but included in the chart.

FIG. 10I depicts a comparison figure of TBR measurements and pre-operative PET-SUV values. Representative CT and PET images are provided. IMI images are displayed on the right panels with white light (visible light) and NIR overlay (IMI fluorescence detected by a camera system). FIG. 10II depict representative comparative images between the calculated 1st and 3rd quartiles in situ TBR values for patients diagnosed with malignant adenocarcinoma. Pre-operative CT scan of the lesion of interest are shown in the panel marked “A” and the panel marked “F”, intraoperative real-time white light images are shown in the panel marked “B” and the panel marked “G”. Once the NIR laser is turned on, the representative NIR images of the lesions are shown in the panel marked “C” and the panel marked “H” with appropriate areas depicted for tumor to background ratio calculation. The overlay images in the panel marked “D” and the panel marked “I” have similar intensity and easily identify the area of fluorescence and presumed tumor location. Subsets depicted in the panel marked “E” and the panel marked “J” confirm dye accumulation using fluorescence microscopy. FIG. 10III depict representative comparative images of similar in situ TBR values for patients diagnosed with malignant adenocarcinoma and benign granuloma. Pre-operative CT scan of the lesion of interest are shown in the panel marked “A” and the panel marked “F”, intra-operative real-time white light images are shown in the panel marked “B” and the panel marked “G”. Once the NIR laser is turned on, the representative NIR images of the lesions are shown in the panel marked “C” and the panel marked “H” with appropriate areas depicted for tumor to background ratio calculation. The overlay images in the panel marked “D” and the panel marked “I” have similar intensity and easily identify the area of fluorescence and presumed tumor location. Subsets depicted in the panel marked “E” and the panel marked “J” confirm dye accumulation using fluorescence microscopy.

FIGS. 11A-11C depict correlation of in situ TBR values with tumor differentiation, final pathology, and tumor subtype presented in various boxplots.

FIG. 12 depicts a pictorial representation of lack of correlation of TBR with folate receptor alpha (FRa) expression. The top row shows no tumor fluorescence with 2+ FRa expression, the middle row displays TBR of 3.4 with only 1+ FRa expression, and the bottom row displays lower TBR calculation with higher FRa expression.

FIG. 13 depicts a schematic block diagram of a smart device of the present disclosure.

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Cancer is the second leading cause of death globally and is responsible for an estimated 9.6 million deaths in 2018. The burden of disease on the individuals, community, and the economic implications are significant. In fact, cancer patients had nearly four times higher mean expenditures per person than those without cancer. These differences were larger among individuals aged 18 to 64 years than those older than 65 years, and lung cancer accounted for the largest expenditure out of all the solid organ malignancies. Medicare was the largest source of payment for cancer patients, especially among those older than 65 years. These costs are expected to increase over the coming decades given the aging population demographics of the country.

Despite the unprecedented advances made in cancer immunotherapy over the last decade, optimal therapy that has been proven to offer best disease free, recurrence free, and overall survival has been surgical management. This is particularly true for solid organ malignancies, particularly lung cancer which is the number one cause of cancer related mortality in the world and the U.S. However, surgery for many malignancies is marred with inherent difficulties such as difficult anatomic localization, inability to detect the primary tumor visually, inability to detect adequacy of tumor margins leading to recurrence, and presence of synchronous lesions which were missed by conventional pre-operative evaluation. Complete negative margins are the best predictor of patient prognosis and the above-mentioned challenges have profound impact on management of these patients.

To address these problems in surgical oncology, Intra-operative Molecular Imaging (IMI) also known as Fluorescence Guided Surgery, has played an integral role. This technology involves injection of an inert NIR tracer systemically which can be selective or non-selective for specific tumor tissue which are then excited and visualized using specialized camera systems. Since the tracers are in the NIR range, they would be otherwise not visible to the human eye. However, given the heterogeneity in the local tumor environment, patient characteristics, and inherent tumor biology, the information provided by the fluorescence of the tumor tissue are difficult to interpret. Certain tumors may produce higher fluorescence compared to others. Inflammatory tissues and benign lesions produce background autofluorescence that obscures findings and lead to more than necessary parenchymal resection. On the other hand, certain fluorescence patterns can be characteristic for definite tumor tissues and that would eliminate extra wait time in the operating room for the surgeon as current standards require the pathologist to confirm malignancy which can take over 30 minutes. The current process of waiting for pathologic confirmation is not without issues as each additional minute is ultimately burdening the hospital and patient with major financial consequences.

### Smart Device for Detecting Malignancy

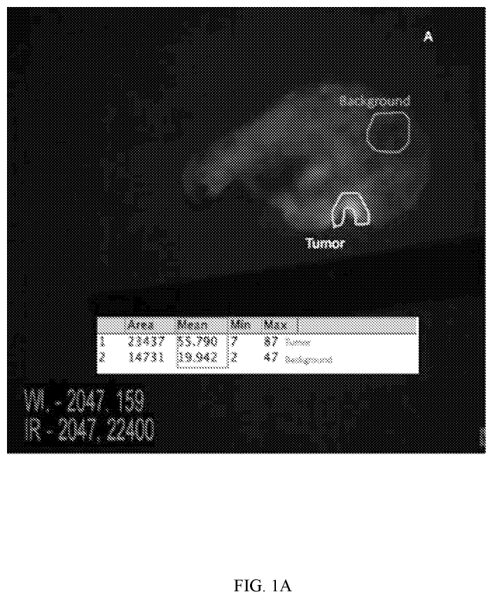

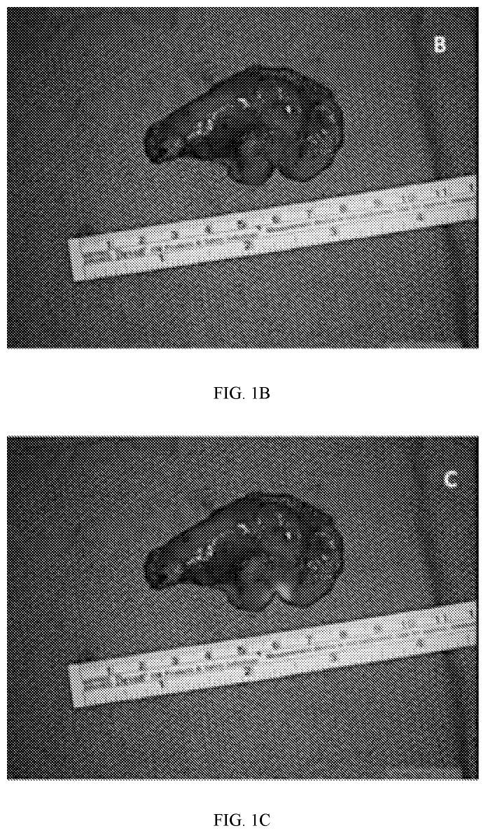

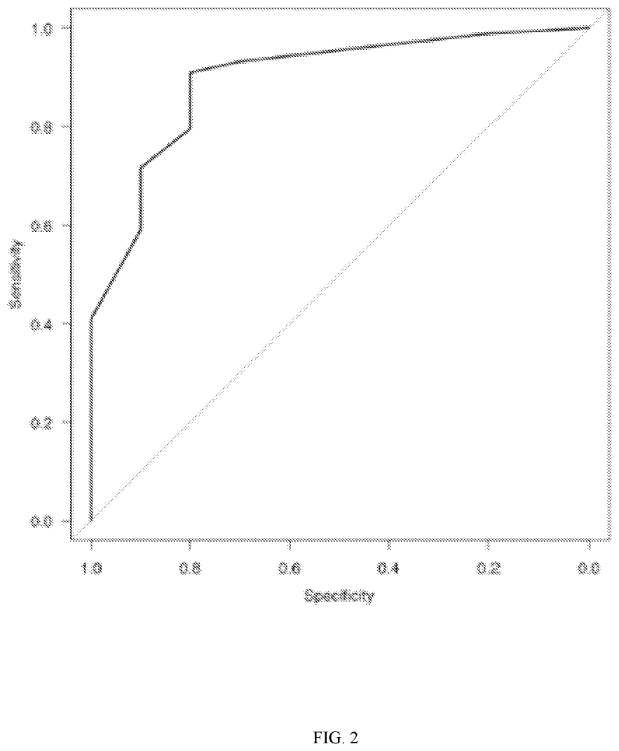

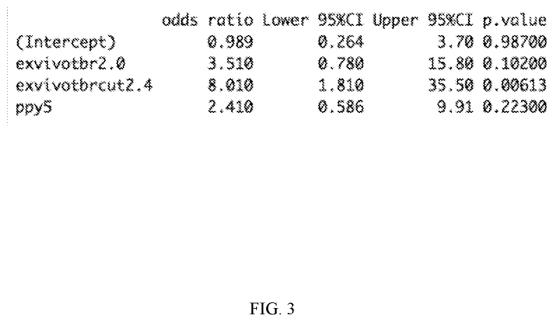

According to embodiments described herein, a smart device that can utilize optical imaging and quantify background and tumor fluorescence in real time based on predictive models is described herein. The smart device can assess the probability of the lesions being malignant or not in the operating room without waiting for significant time (e.g., over 30 minutes) for confirmation. This device can include additional instruments, such as a light source, camera optics, and computing device, which can reliably detect the likelihood of malignancy in real-time of any fluorescent tissue. Prototype algorithm findings are presented below (FIG. 2). Additionally, the device can automatically calculate areas of NIR fluorescence which are conventionally performed by hand (FIGS. 1 and 3).

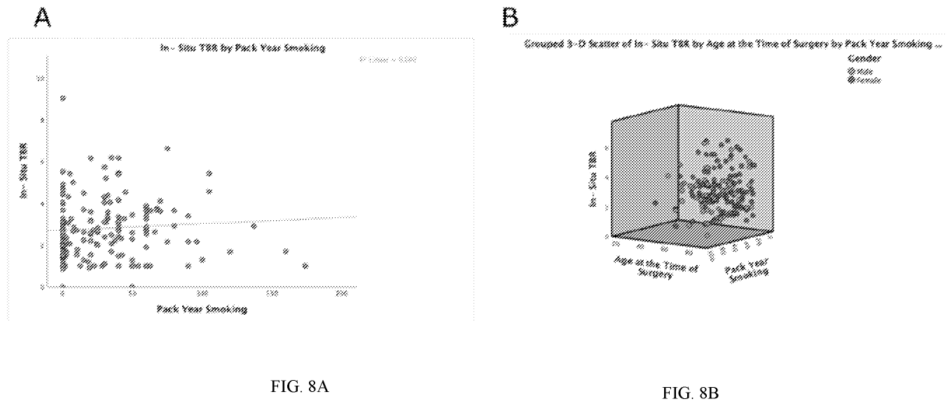

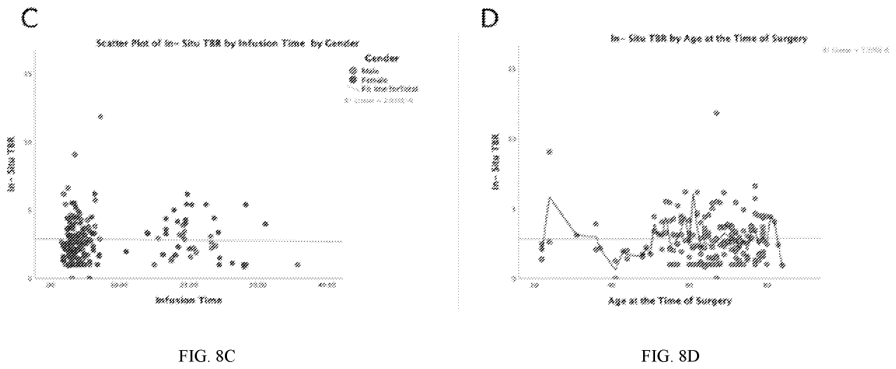

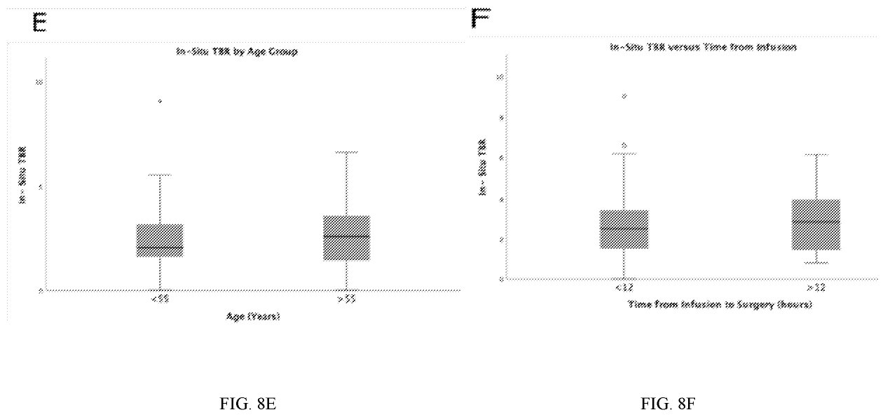

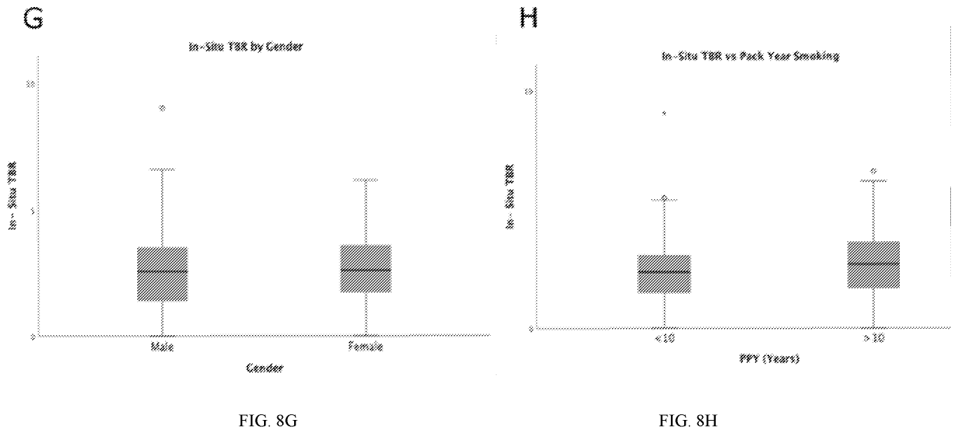

The smart device can predict the likelihood of malignancy in a multitude of ways. First, if the patient has been injected with an optical, fluorescent probe, then an imaging system can detect the lesion using an optical sensor and alert the surgeon that fluorescence has been detected and based on pre-operative patient characteristics alert the likelihood that this tissue corresponds to concerning lesion. Second, once the tissue fluorescence is identified, the device can quantify tumor and background fluorescence using artificial intelligence which is then aggregated with patient demographics; and subsequently display a probability of a lesion being cancer on a screen (FIG. 8D). Thus, a surgeon can proceed with appropriate surgery rather than resect the lesion and await tissue confirmation.

The smart device can utilize semantic image segmentation to identify the boundaries of the organ relative to other portions of the image (e.g., a surgical table). Additionally or alternatively, a medical professional can draw the approximate boundaries using an input device such as a stylus, a mouse, a touchscreen, and the like.

Finally, upwards of 25% of patients with solid organ malignancies can present with synchronous lesions or advanced disease that are not appreciated on pre-operative high-fidelity imaging. The smart device can detect these organ malignancies and alert the surgeon that the patient requires additional surgical resection. Use of the smart device can therefore lead to a decrease in malignancy recurrence and/or point the patient to more aggressive systemic therapy, which can lead to better long-term outcomes and survival.

The consequences would be providing faster, less expensive, less subjective, and less labor-intensive data to the surgeon, which has major implications for a given patient

For example, for lung cancer operations in the United States, most patients are presented with a pre-operative imaging study (e.g., CT, MRI or PET/CT) that is suspicious of pulmonary malignancy but with no confirmed pre-operative diagnosis. The surgeon will proceed with the operation and will generally take a diagnostic wedge resection of the lesion to confirm or deny the presence of cancer. This wedge specimen is sent from the OR directly to the pathology department for a frozen section diagnosis. Meanwhile, the operation is effectively put on hold, while the patient is under anesthesia, pending the results of this diagnostic section. Wait times at hospitals can average over 26 minutes. During those 26 minutes, the pathologist or their assistant locates the lesion within the tissue wedge, excises the tissue of interest and embeds it in a cutting medium for freezing. They proceed to cut through the embedding medium and tissue until they determine they have reached the optimal, diagnostic area of tissue. They then prepare a microscopy slide with typical biological stains (generally hematoxylin and eosin stains, or “H&E”, for example) for analysis by the pathologist. Once a diagnosis has been determined, the pathologist relays the information back to the operating room, where the surgeon can finally proceed with the operation according to the standard of care: either terminating the case or removing additional lung tissue.

### Optical Imaging and Real-Time Analysis of Optical Imaging Data

Optical imaging is the technique used to excite fluorophores using an optical wavelength light source and then capturing the resultant narrow emission using an optically sensitive camera. These fluorophores can be conjugated to molecules that specifically target and attach with high affinity to different types of cancers, inflammation, and bacterial infections, among other diagnoses. For cancer surgery, fluorophores that preferentially target the tumor can be excited in real time to illuminate cancer that would be difficult or impossible to locate by feel or the naked eye alone.

The devices and systems described herein can include a model for detecting malignancy in a patient tissue from images taken of the patient tissue. The model has been validated in various widely available NIR tracers, which produce fluorescence at a range of approximately 800 nm. Malignant tissues produce characteristic fluorescence which is currently calculated as Tumor to Background Ratio (TBR), which takes the ratio of mean fluorescence intensity of the tumor to mean fluorescence intensity of the normal background.

Different TBRs can be calculated and compared to different references to reflect the effect of the environment in which the TBR is calculated. For example, the TBR for an image calculated inside the body (e.g., inside the chest) before removal of a volume of concern may be different than a TBR for an image after resection but before removal from the body or a TBR for an image after removal from the body (at which point the operating room lightings will influence the image).

Optical imaging has been shown to effectively localize cancer nodules as small as approximately 2 mm within surrounding normal tissue in real-time during surgery. Because these fluorescent dyes can be targeted to cancer-specific receptors, this technology can augment the gold standard of pathological evaluation of frozen sections through the “optical biopsy.” If a piece of tissue fluoresces when exposed to a fluorescent tracer, the tissue is highly suspicious for cancer, specifically adenocarcinomas of the lung. In much the same way as a PET scan gives additional information to the surgeon prior to the operation, the techniques of optical imaging- and real-time analysis of frozen sections-gives the surgeon additional information when making his/her diagnosis.

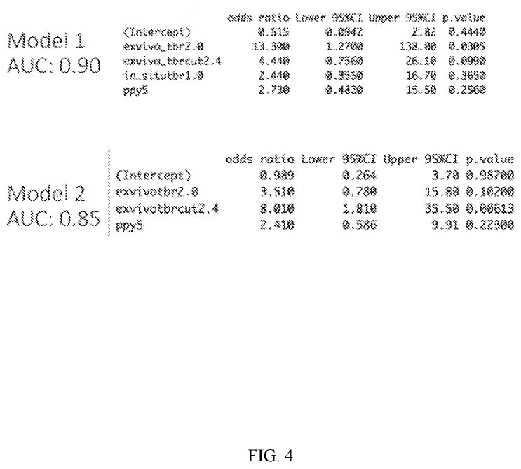

However, not every fluorescence is foretelling. For example, TBR of 2.1 on a young patient who is a nonsmoker is not the same as TBR of 3 in a 65-year-old with smoking history. Building patient demographics and statistics on operating on patients with lung lesions over the last 5 years, data from over 300 lesions with complete pre- and post-operative follow up data was collected and utilized to generate a high-fidelity algorithm. A group of 225 patients was selected to serve as a test set. Twenty-two (22) distinct parameters were selected from the database including pre-operative imaging, demographics, co-morbidities, as well as robust peri-operative tumor characteristics including fluorescence patterns noted during intra-operative molecular imaging guided surgery. Using logistic regression analysis, multiple models were generated and assessed for predictive capability of the algorithm for malignancies. The results showed that the models with highest predictive reliability had areas under the curves of 0.897 and 0.85 on receiver operator curves which are very strong (FIGS. 2-4). The accuracy of the algorithms were confirmed on the validation set which demonstrated excellent concordance indexes.

### Artificial Intelligence Implementation

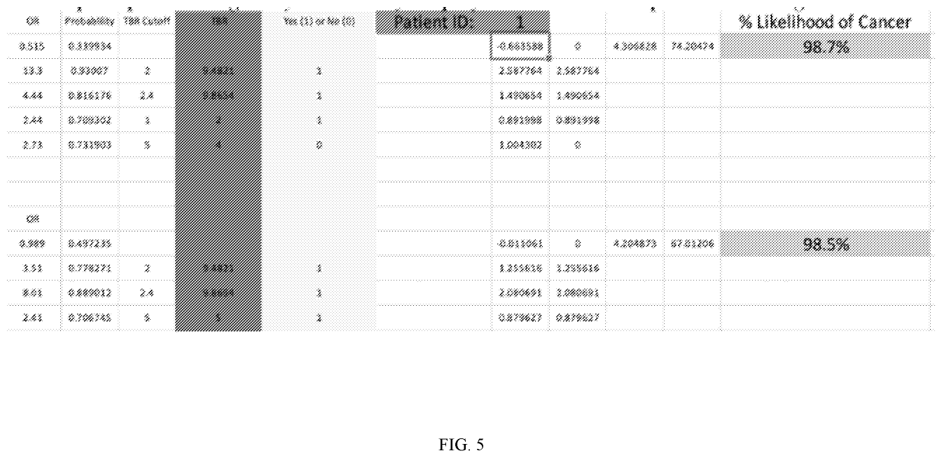

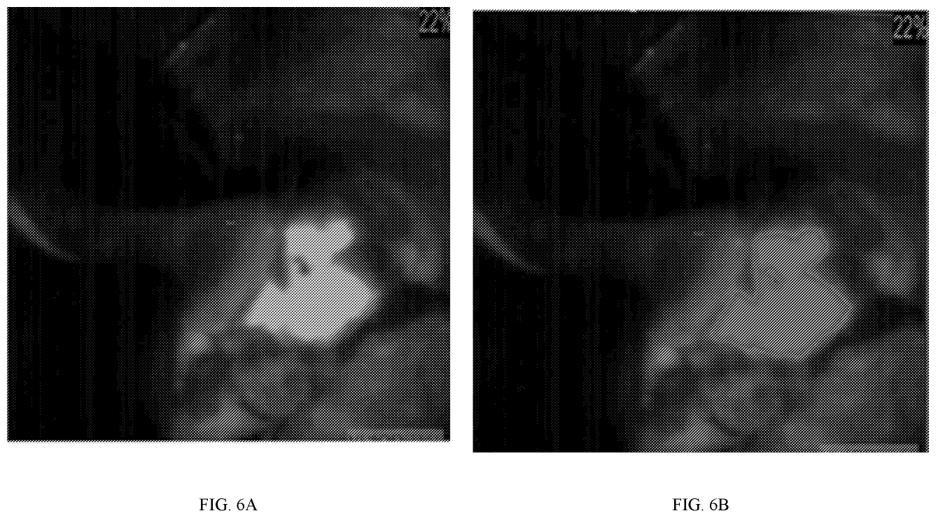

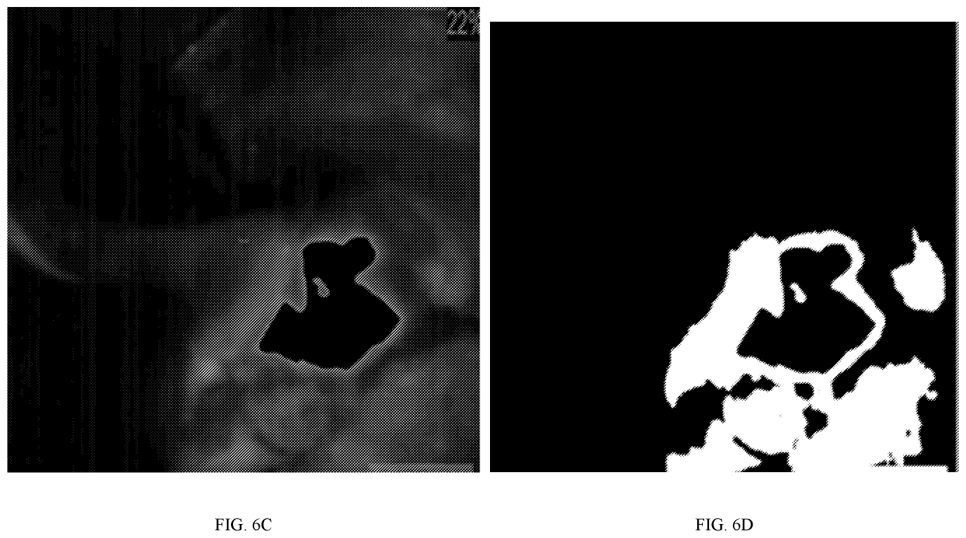

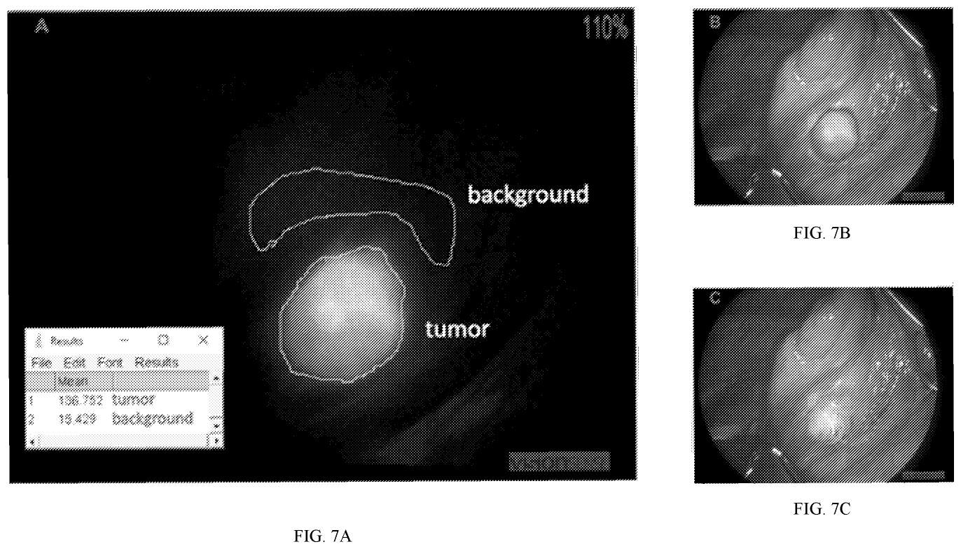

The devices, systems, and methods described herein can also utilize an artificial intelligence functions training and improving the underlying malignancy detection models. The proposed algorithms can be implemented, for example, as a MATLAB function (using MATLAB R2012a) in conjunction with Simulink, Bioinformatics toolboxes, communications toolboxes, computer vision toolboxes, data acquisition toolboxes, deep learning toolboxes, fuzzy logic toolboxes, image acquisition and image processing toolboxes, predictive maintenance toolboxes, machine learning toolboxes, and the like. Several ranges of values with optimal cut off values can be deduced from the statistical models which can predict or negate presence of malignancy in tissues in a matter of seconds. To further improve and standardize measurements for the smart device and related methods, deep learning and computer vision toolboxes (e.g., from MATLAB) can be utilized. Fluorescence intensity, which is what ultimately predicts TBRs, can be calculated manually (FIG. 1). Invariably areas that are selected for calculation are subject to operator bias and might not reflect accurate tumor or background area. Further, each normal parenchyma has their own autofluorescence; inaccurate identification or misinterpretation of normal fluorescence patterns might falsely point the researcher towards an inaccurate diagnosis. The smart device and related methods described herein eliminate this process by training the deep learning toolbox in the coding algorithm to identify normal background tissue and tumor location, which then produces a standardized pattern of fluorescence quantification, which is not subject to observer or researcher bias. For example, using a Fuzzy Box Logic toolbox, quantified values can be entered into the predictive model and results can be seamlessly displayed for the surgeon to act on within 10 seconds (FIGS. 5 and 6). A surgeon can then proceed with standard of care surgery rather than wait upwards of 30 minutes for pathology frozen section confirmation which would subject patients to additional unnecessary anesthesia. In essence, the surgeon has to point the device at an area that he/she finds concerning and within seconds will get accurate assessment of malignant potential of that tumor.

In some cases, the machine learning algorithms can be operated across multiple platforms. For example, the machine learning algorithm can be supported by iOS, Raspberry Pi, Android, and the like. In some cases, the machine learning algorithm can also include other processes for refining the training, analyzing, and determination processes regarding malignancy. For example, the machine learning algorithm can also include standardization processes for refining the training model. Further, the processes of the machine learning algorithm can minimize or eliminate bias or inaccuracies from manual TBR calculations.

### Device and System

The smart device described herein can include a singular device capable of performing the processes described herein. For example, the device can include an optical imaging system capable of detecting NIR fluorescence dye injected into a patient. The device can include processors and memory capable of performing the TBR analysis for images captured by the optical imaging system, such as delineating areas of high fluorescence, areas of low fluorescence (e.g., on a pixelated level), and calculating a TBR from this delineation. The smart device can also include the algorithms capable of identifying malignancy based on the TBR and patient demographics.

In other cases, the processes described herein can be performed by a system. The system can include an optical imaging system, which can be coupled (e.g., wired or wirelessly) to a computing device(s) capable of performing detecting the fluorescence, determining the TBR, and determining the probability of malignancy. The computing devices can either be personal computing devices, or can be other computing devices such as servers, databases, cloud computing, and the like.

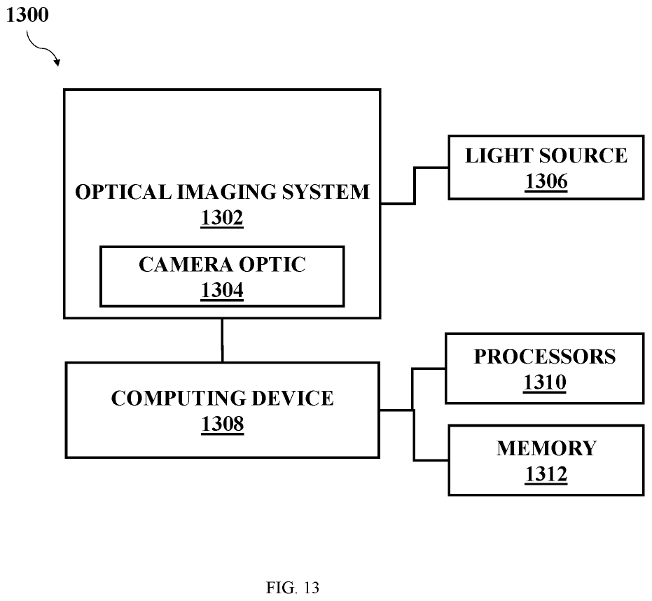

Certain embodiments of the device can be described in connection with the drawings. For example, FIG. 13 illustrates a smart device 1300 configured to implement certain methods of the present disclosure. Smart device 1300 includes an optical imaging system 1302 including a camera optic 1304 configured to image at least a section of a tissue of a patient. Optical imaging system 1302 can be configured and adapted to detect near infrared (NIR) fluorescence dye injected into a patient. Smart device 1300 includes a light source 1306 configured to illuminate a section of tissue of the patient. Smart device 1300 includes a computing device 1308 configured and adapted to utilize optical imaging and quantify background and tumor fluorescence in real time based on predictive models. Computing device 1308 can be configured and adapted to assess the probability of lesions being malignant. In certain embodiments, smart device 1300 can further include processors 1310 and memory 1312 capable of performing TBR analysis for images captured by optical imaging system 1302, where the processors 1310, memory 1312, and/or TBR analysis can delineate areas of high fluorescence, delineate areas of low fluorescence, and calculate a TBR from delineating the areas of high fluorescence and the areas of low fluorescence.

### Patient Tissue

The processes performed herein can be conducted on a multitude of patient tissues. Some of the examples provided herein are conducted on patient lungs. However, the processes can be performed on various other organs as well (e.g., liver, stomach, kidneys, and the like). The patient tissue can also be lymph nodes, glands, connective tissues, and the like. Further, the processes performed herein can be conducted both in vivo and ex vivo. In some cases, the processes can be performed on bisected tissue. In some cases, the process can be performed multiple times on a patient tissue. For example, images can be captured and analyzed first in vivo, and the process can be performed again ex vivo.

### Process

A process for detecting malignancy in a patient tissue is described herein. The process can be implemented by a smart device or system as discussed above. In step 1, a patient tissue can be injected with a dye. The optical dye can be injected into the patient before the patient tissue is removed. This would allow localization to any tumor cells in the patient tissue (e.g., an organ or lymph nodes). Thus, the tumor can be fluorescent (e.g., to the assisted eye using an optical camera).

At step 2, a user (e.g., a surgeon or other healthcare professional) of an optical camera system can position the optical camera system to image the patient tissue. The optical camera system can detect the fluorescent dye in the patient tissue. The smart device can receive the images taken by the optical camera system and quantify fluorescence of any tumor and the background. Calculated values can be entered into the algorithm and a probability of cancer can then be displayed to the user in real time.

At step 3, a computer algorithm can advise the user on potential malignant tumor probability. The computer algorithm can calculate this probability from the TBR of the received images, information received from the patient (e.g., demographic information, and the like), and the trained model (e.g., previously determined malignancies from previous patients and their respective patient demographics). In some cases, the computer algorithm can notify the user that an additional lesion is detected and recommend resection (e.g., from the received camera images, patient demographics, and trained model).

### Advantages

Currently, there is no technology available for providing the surgical oncologist about the malignant potential of the tissue in real time during fluorescence guided surgery. The smart device described herein can instantly detect the lesion, quantify its fluorescence, and predict likelihood that it is a malignant tissue. The surgeon then can act on that information by proceeding with standard of care surgery or abandon the procedure. The patient no longer has to wait upwards of 30 minutes under anesthesia and the time saved can additionally free up operating room resources for additional cases, which has serious financial repercussions. Unlike other imaging modalities, the smart device does not require ionizing radiation. The energy to excite fluorescent contrast agents is low (e.g., 101 eV) and is less than the lights emit in an operating room, for example. Furthermore, the smart device is intuitive because it is visual and does not require special training or experience. Real-time molecular imaging does not require advance knowledge of the location of the primary nodule or metastases. Optical imaging is easy to understand and can image large surfaces real-time without disrupting the natural flow of the operating room. Lastly, the smart device can reliably and consistently calculate fluorescence of the tissues as it uses an algorithm and won't be subject to observer bias.

### EXPERIMENT 1

### Introduction

Cancer surgery has multiple challenges including localizing small lesions, ensuring negative surgical margins, and identifying potential synchronous cancers. One of the tools proposed to address these issues has been intraoperative molecular imaging (IMI) or fluorescence-guided surgery. IMI utilizes a systemically injected fluorescent optical tracer that preferentially accumulates in abnormal lesions and can be visualized using frequency-specific camera systems. Utilization of this technology has been found to be safe and promising in a number of oncologic procedures, including neurosurgical, gastrointestinal, thoracic, and soft-tissue malignancies. Innovations in IMI have been key in providing real-time feedback on nodule location and size without compromising patient safety.

An important consideration in optical molecular imaging during surgery is the quantification of the tumor fluorescence during an operation and the clinical significance of that quantification data. Currently, the most commonly cited measure of quantification is the tumor-to-background ratio (TBR). Other synonymous terms include signal-to-background or signal-to-noise ratio. TBR calculation takes the ratio of mean fluorescence intensity (MFI) of the tumor at a selected location (xₜ, yₜ, zₜ) to the MFI of the background un-diseased tissues such as the adjacent organ, adipose tissue, or muscular tissue (x_{b}, y_{b}, z_{b}). TBR often is calculated using various computer algorithms, though, the most cited is ImageJ because it is freely available from the National Institutes of Health. The TBR is frequently presented as:

$$TBR ( x , y , z ) = \frac{MFI Tumor ( {x}_{t} , {y}_{t} , {z}_{t} )}{MFI Background ( {x}_{b} , {y}_{b} , {z}_{b} )}$$

The clinical significance of the TBR has not been rigorously studied. Much of the clinical utility of TBR is drawn from parallels in medical nuclear imaging, specifically, positron emission tomography (PET). In PET, the standard uptake values (SUVs) measures relative uptake of the radioactively tagged glucose in a tissue/organ relative to the background. An SUV of two or greater has been shown to have some clinical value because it can predict the malignant potential of undiagnosed nodules in colon cancer, non-small cell lung cancer, pancreatic cancers, as well as various head and neck cancers.

Despite the increase in clinical trials, fluorescent tracers, and optical imaging devices, a standardized approach to image analysis using TBR has been lacking. This is partly due to the fact that each cancer is vastly different owing to unique normal background selection, tumor metabolism, and tracer selection, all of which can influence TBR calculations (Table 1). Nevertheless, the most commonly cited value that has been referenced as being clinically significant in optimal imaging has been a TBR>2 to represent a positive event. Analyzing these variations, previous research has concluded that the cutoff value of TBR>2 was based on marginal evidence and urged caution in using cut-off values for clinical decision making.

TABLE 1
Common factors implicated in TBR variability in the literature:

Tracer to tumor specificity
Tracer pharmacodynamics and pharmacokinetics Optical camera
sensitivity
Background tissue metabolism and noise
Tracer optical qualities (scattering, absorption, autofluorescence,
depth of penetration)
Post-image processing (selection of appropriate tumor and
background, image quality, software used)

The purpose of this study was to determine whether TBR measured during OTL38 based IMI-guided lung resection can provide clinically useful information. Specifically, for this study, the “in situ” TBR was analyzed for a single tracer for a single indication to create a consistent model. Several variables were considered, such as malignant potential of the tumor (based on pre-operative investigation such as social history, imaging findings, and other clinical risk factors), intrinsic tumor characteristics (tumor differentiation, type, and tumor subtype), as well as final histopathologic diagnosis and then determined if these variables were correlative with in situ TBR measurements.

### Materials and Methods

### Data Collection

Appropriate authorization was obtained from the University of Pennsylvania Perelman School of Medicine Institutional Review Board prior to data collection. Data was retrospectively analyzed from a prospectively collected database. Surgical procedures were performed by the surgeons at the Department of Thoracic Surgery at the Hospital of the University of Pennsylvania. A total of 279 patients were included in the study. All patients enrolled in the study presented with suspicious pulmonary nodules in conjunction with the recommendations from the Fleischner Society Guidelines for Pulmonary Nodules based on high-quality 1 mm slice thickness CT scanning. Based on institutional practice patterns, pre-operative tissue sampling and diagnosis was not mandatory. Selected patients underwent appropriate pre-operative clearance and metastatic disease work up prior to operative scheduling with video-assisted thoracoscopic surgical (VATS) lobectomy being the procedure of choice.

### Surgical Procedure and NIR Tracer

Analyzed patients underwent infusion of the study drug OTL38 (0.025 mg/kg) up to 24 h prior to surgery. OTL38 (C₆₁H₆₃N₉Na₄O₁₇S₄, MW: 1414.42) is a folate analog conjugated to the NIR dye, S0456. OTL38 excites at 774-776 nm and emits at 794-796 nm. Specifically, OTL38 targets folate receptor alpha which is expressed in more than 90% of adenocarcinomas of the lung. During pulmonary resection, surgeons utilized white-light (bright field imaging) and finger palpation through port site incisions to confirm the lesions in the anatomical location of interest. In situ, real-time fluorescent imaging was performed using an Iridium (Medtronic, Minneapolis, MN) imaging system optimized for detection of NIR tracers. IMI-based optical devices are high definition, dual-band (white light and NIR) camera systems capable of concurrent NIR emission and detection while generating real-time video. The iridium system utilizes an excitation laser with a wavelength of 805 nm, with fluorescence detection based on a bandpass filter selective to light ranging from 825 to 850 nm. During VATS, systems were equipped with NIR-calibrated thoracoscopes.

### Specimen Analysis and TBR Calculation

Postoperatively, all the IMI images were uploaded to the deidentified secure database for analysis. Mean fluorescence intensity of the tumors was calculated from ImageJ (from National Institutes of Health) software with minimum 1000 pixels being included in the analysis. To minimize inter-observer bias, a protocol was established where the in situ and ex vivo TBRs were calculated by three blinded independent observers. Results were re-calculated by different observers if at least two blinded observer TBR measurements were not correlating (TBR values ±0.2 from each other). TBR results were then compared to final pathological diagnosis and analyzed for their correlation with in situ fluorescence. Additionally, correlative analyses were performed for TBRs with final pathological diagnosis for values in the first and third quartiles.

### TBR Analysis

To evaluate the efficacy and variability in TBR calculation, patient factors (age, gender, time from infusion to surgery), biologic factors (presumed clinical malignant potential such as depth of lesion as measured on pathology, size of the lesion as measured on cross sectional imaging, location of the lesion, correlation with PET SUV), tumor characteristics (malignant vs benign, tumor type, tumor subtype, differentiation), and technological factors with respect to in situ TBRs were analyzed.

Both parametric and non-parametric statistical analyses were performed using R version 3.5.3 as well as SPSS version 27 (IBM Technologies). P values <0.05 were considered statistically significant.

### Results

### Demographic Factors

To evaluate the hypothesis that intrinsic patient factors alter in situ TBR measurements, correlative analysis for in situ TBR was performed for patient age, patient gender, interval time from infusion to surgery, and pack year smoking history. A total of 279 patients were included in the final analysis. The average age at the time of surgery was 64 years and 60% of the patients were female. All patients underwent infusion of the study drug within 24 h of scheduled surgery with mean time of 7.7 h from infusion to surgery. Median American Society of Anesthesiologists (ASA) class for the entire cohort was three with median length of stay (LOS) of 2.2 days. There were no significant complications related to IMI tracers (Clavien-Dindo 9III) encountered in the analysis. Details are summarized in Table 2.

TABLE 2
Patient Demographics for 279 Patients Analyzed in the Study

| | Mean | Std. deviation |
|---|---|---|
| Age at the time of surgery (years) | 64.0 | 12.8 |
| Time from infusion to surgery (hours) | 7.7 | 8.3 |
| Pack years smoking (years) | 30.5 | 35.1 |
| Distance from pleural surface (cm) | .37 | .66 |
| PET SUV | 4.4 | 5.1 |
| Tumor size (cm) | 1.9 Median | 1.4 |
| ASA classification | 3 | |
| Length of stay (days) | 2.2 | 0.7 |

Analysis of patient factors that could alter in situ TBR measurements during VATS for pulmonary lesions did not show statistically significant correlation with gender, age, smoking history, and time from infusion to surgery with respect to in situ TBR calculations (FIGS. 7A-7C). Furthermore, no statistical correlation (p=0.213) was noted between patient factors and the surgeon identifying the lesion of interest as long as it fluoresced in situ (TBR>1.1). Subgroup analysis of the variables in each quartile yielded similar lack of correlation (FIGS. 8A-8H).

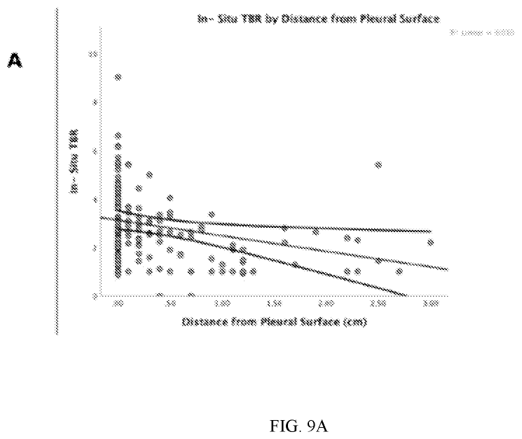

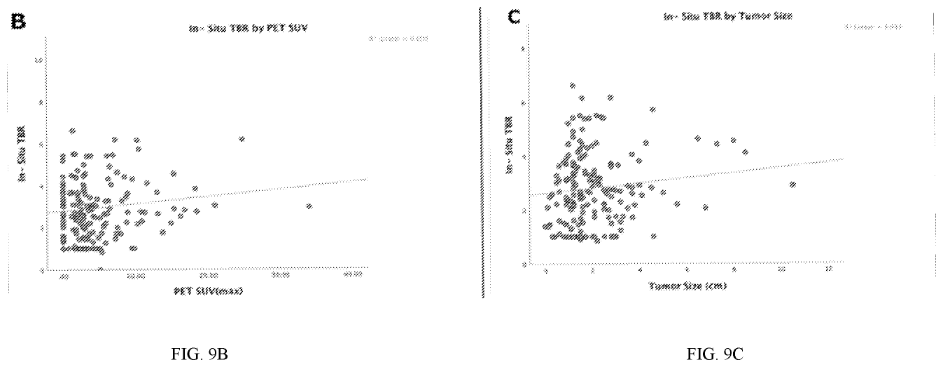

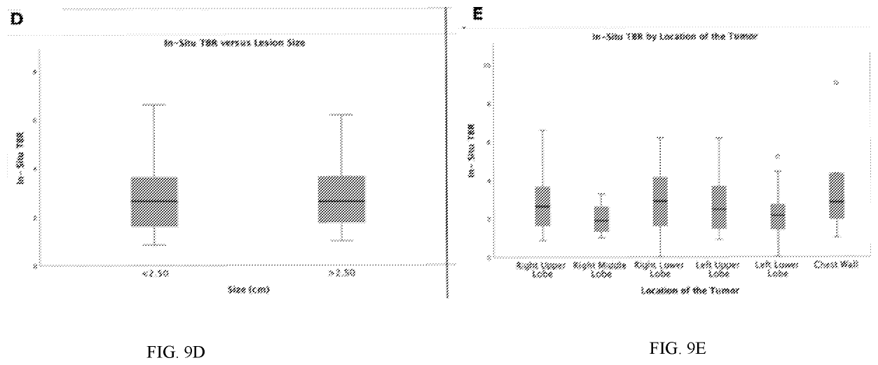

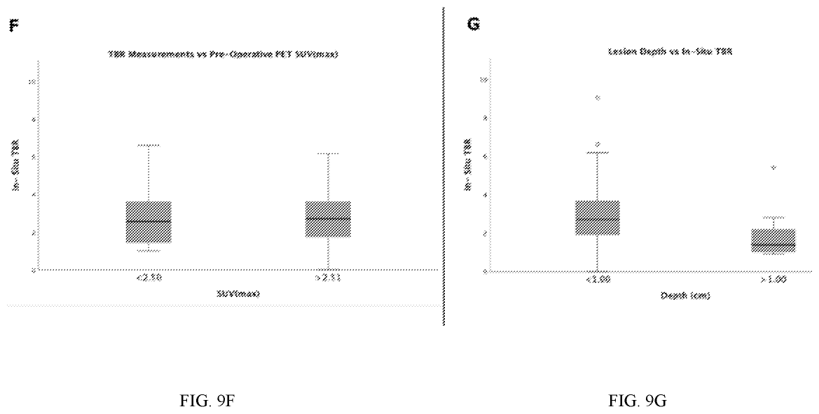

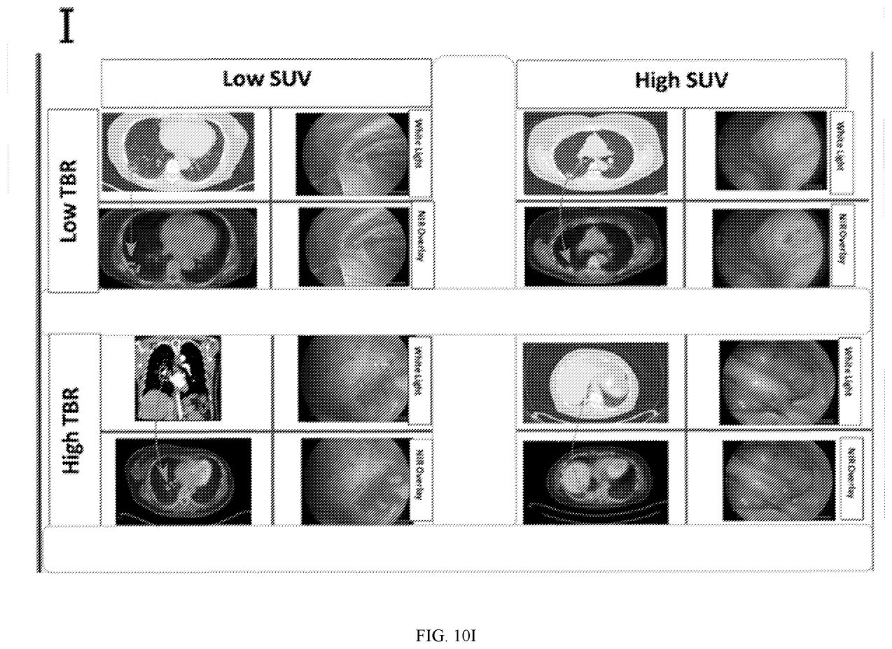

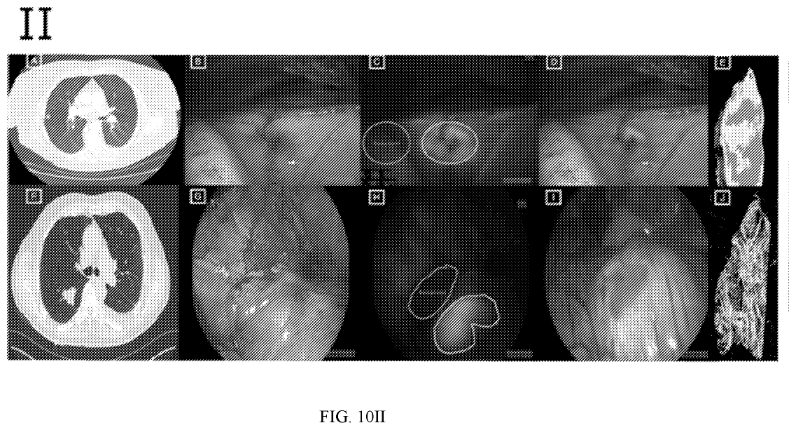

Pre-operative Biological Factors: In order to determine whether biological variables predict intraoperative TBR, the following tumor characteristics were compared to intraoperative fluorescence: depth of the lesion on pre-operative cross-sectional computed tomography imaging, positron emission tomography standardized uptake value (SUV), location of the lesion, and size of the primary lesion. There was no statistical predilection for tumor location in our cohort based on location of the lesion (p=0.698). Table 2 summarizes the biologic characteristics in the patient cohort which shows mean tumor size of 1.9 cm, depth of 0.37 cm, and mean SUV (max) of 4.4. TBR measurements did not correlate with location (p=0.123). Additionally, there was no statistical correlation of SUV (max) values with measured TBRs in-situ (FIG. 9). As demonstrated in FIG. 10I, lesions with high or low pre-op SUV did not predict if the lesion would fluoresce.

On the other hand, there was statistically significant correlation of in situ TBR measurement and depth of the lesion from the surface of the lung. Deeper lesions were negatively correlated with TBR measurements (p<0.01). Statistically significant variation with size and the TBR calculation in situ was not observed. Overall, regardless of tumor size and pre-operative PET imaging, lesions on the pleural surface fluoresce under the right circumstances with decreasing likelihood at depth greater than three cm as there was minimal fluorescence observed for lesions beyond this depth. Results are summarized in scatter plots in FIGS. 9A-9C with associated fitted regression line.

To further explore possible correlations of pre-operative biological factors, values of SUVs, size, and depth were binned into subgroups based on quartiles. Again, there was no statistically significant correlation with the exception of depth, which is in line with previous reports in literature. Results are summarized in FIGS. 9D-9G. All the lesions that fluoresced (TBR 9 1.1) were identified and removed by the surgeon regardless of location, size, and pre-operative imaging.

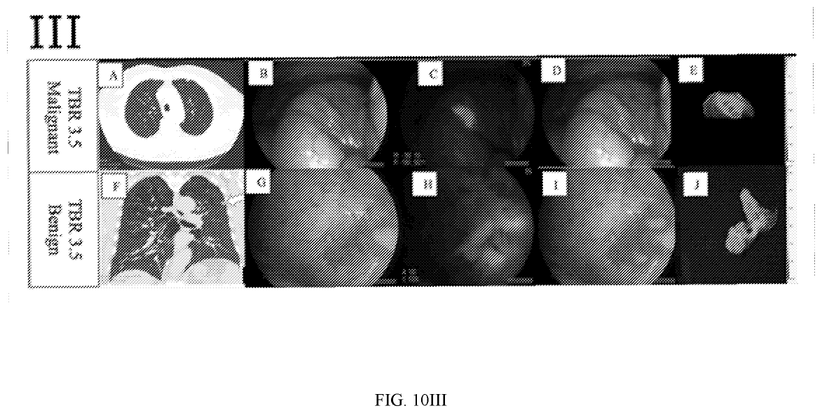

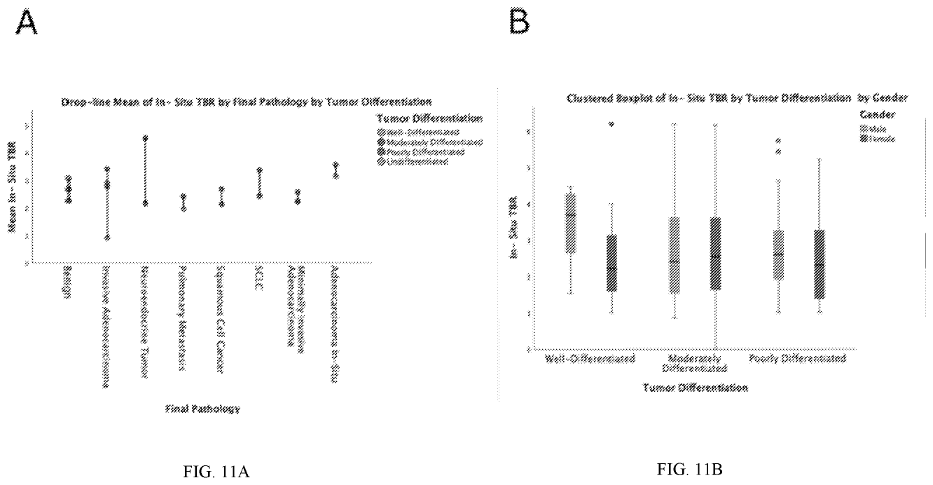

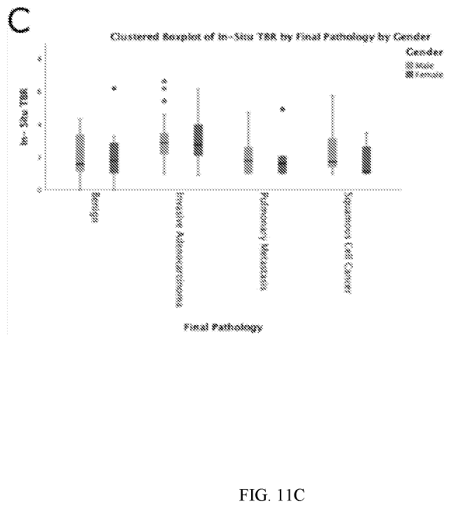

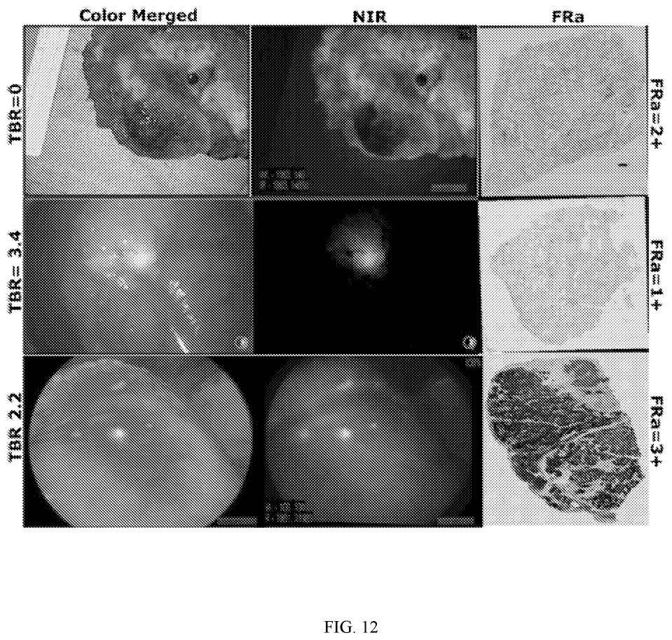

Intrinsic Tumor Characteristics: Given that patient intrinsic factors and pre-operative biological factors did not correlate with intraoperative TBR, it was postulated that tumor biological factors may predict the TBR. In situ TBR values were compared to final tumor histopathology, tumor subtype, and tumor differentiation (FIGS. 11A-11C).

As predicted from pre-operative patient selection criteria, the most common final pathological diagnosis was invasive adenocarcinoma (50%) with adenocarcinoma spectrum lesions compromising more than 60% of patients in the cohort. Additionally, 14.3% patients had benign lesions which accounted for second largest final pathological diagnosis. More than 70% of benign lesions were granulomas. The majority of the patients had either poorly or moderately differentiated tumors with most patients having acinar subtype tumors. Details of tumor characteristics are displayed in Table 3.

Adenocarcinoma spectrum lesions overall had statistically significant correlation with in situ fluorescence and in situ TBRs compared to other NSCLC malignancies (p<0.01) but TBR measurements could not identify histopathologic subtype on univariate analysis (p=0.089). There was a tendency for in situ fluorescence for moderately and well-differentiated adenocarcinoma spectrum lesions (FIGS. 11A-11C), but this was not statistically significant on analysis of variance for TBR measurements when comparing values for each subgroup (well-differentiated, moderately differentiated, poorly differentiated) (p=0.061). Additional subgroup analysis looking at gender distribution with respect to tumor differentiation, final pathology, and tumor subtype did not reveal correlation with in situ TBRs. Graphical representation is provided in the clustered boxplots in FIGS. 11A-11C.

While there was observed association of in situ fluorescence with certain histopathologic diagnoses, further analysis was performed to characterize the association of pathology with in situ TBR measurements. Mean calculated TBRs for in situ, ex vivo, and bisected specimens were 2.84, 3.09, and 4 respectively (Table 4). When analyzing in situ TBR values with major final histopathologic diagnoses, invasive adenocarcinomas had the highest average TBRs (p<0.05) compared to other malignant lesions. On univariate analysis, invasive adenocarcinomas were correlated with higher average TBRs compared to other malignancies. However, these associations were not evident on multivariate analysis. When comparing mean in situ TBRs of benign lesions to in situ malignant TBRs, there were no statistically significant associations. Further analysis of TBR values in the first and third quartiles was performed. For TBR values less than 2 (first quartile), 30 patients were identified. Of these patients, 22 had malignant nodules and all fluoresced intra-operatively.

Final pathology report confirmed adequate margins for all the patients with none of the patients requiring additional resection. Furthermore, analysis of 28 additional patients with TBRs greater than 4.9 (third quartile) who were localized in situ of whom 24 had malignant diagnosis were performed. Both parametric (paired sample t-test) and non-parametric (Mann-Whitney-U test) comparison between the groups did not reveal statistically significant difference in rate of adequate oncologic resection and localization (p=0.145).

A pictorial representation of malignant adenocarcinomas with TBR values in the first and third quartiles is displayed in FIG. 10II. In summary, adenocarcinoma spectrum lesions tend to fluorescence at a higher level compared to other histologic subtypes. However, there are no statistically significant associations of TBR values with tumor types, tumor subtypes, and tumor differentiation (Table 5).

### Discussion

Over the last few decades, the field of surgical oncology has taken significant strides in terms of innovation and patient outcomes. One can argue that IMI has had an integral role in these advancements and will be a vital aspect of oncologic surgery over the coming decades. A key part of evaluating the utility of IMI is the usefulness of the quantitative data of nodule fluorescence that is retrieved during a surgery. A hypothesis during this study was that TBR calculations in situ have clinical utility in informing the surgeon about the malignant potential of the lesion and aggressiveness of the operation. However, it was found that the intraoperative in situ TBR values need to be interpreted carefully in conjunction with other clinicopathologic variables. The true value of IMI was gross detection of fluorescence by the surgeon in real-time for finding the nodule, identifying margins, and drawing attention to synchronous lesions.

TBR measurements are based on dye accumulation, distribution, and likelihood of malignant cell uptake; certain patient factors could skew fluorescence uptake and quantification on above named mechanisms. The variation in situ TBR measurements based on patient age, gender, smoking history, and time from infusion to surgery was examined. Statistically significant contributions from these factors on TBR calculation were not found (FIG. 8). This means that IMI has a broad appeal and efficacy across a diverse demographic and not necessarily reduced to a limited patient population. Once the lesion is identified and deemed to be high enough risk for surgical resection is what appears to be enough for IMI to be employed. The lesions which glowed regardless of in situ TBR values were removed and sent for successful histopathologic examination. This indicates that using visual cues along with tumor specific fluorescence, a majority (if not all) lesions can be removed in an oncologically safe manner regardless of patient demographics. FIG. 10II, illustrates clear difference in fluorescence of different lesions both in NIR and overlay mode in patients with similar preoperative characteristics (gender, age, size of the lesions, depth of the lesion, and smoking history). When this image was shown to other observers without knowledge of the study or the case, all 10 selected the top sub-section as the one with highest TBR. However, the patient in the lower subset (FIG. 10II panels labelled “F”-“J”) has a higher in situ TBR and is visually deceiving for the untrained eye. Therefore, one can argue that a clinician should approach these measurements carefully and use it in conjunction with other pre-clinical, intraoperative, and demographic parameters for optimal oncologic treatment. High TBR in situ with OTL38 during lung cancer resection does not necessarily mean higher likelihood of cancer and vice versa. As evident in NIR microscopy images in FIG. 10II and FIG. III, tumor burden does not necessarily translate into better fluorescence quantification in-situ which can be confounded by patients social determinants (smoking, pollution, occupation), background normal lung parenchymal inflammation, lesion depth, and camera angle.

Not all lesions are optimal for IMI utilization during surgery. Similar to limitations observed in early days of SUV calculation and interpretation, intrinsic characteristics of lesions play a central role in IMI quantification as well. We explored certain preoperative biological factors such as location, PET SUV, and size and depth of the lesion with in situ TBR variation. There was no TBR variation based on anatomic location of malignant lesions. There was also no variation of TBRs with size of malignant lesions as lesions smaller than 2.5 cm or larger essentially had similar TBR quantification. However, there was a statistically significant variation in in situ TBR measurements with depth of the lesion (FIG. 8A). This is a known limitation of IMI and current tracers that operate in the NIR region. Fluorescence based on lesion size and depth has been a topic of debate in the literature in early implementation of IMI but with increasing sophistication of the technology, same quantification can be achieved on a molecular level regardless of size (FIG. 10III, panels labelled “E” and “J”).

TABLE 3
Final histopathological diagnosis in the patient
cohort with associated tumor subtype and tumor differentiation.
Complete details of histopathological breakdown
are provided in supplementary data

| | N | % |
|---|---|---|
| Final pathology | | |
| Invasive adenocarcinoma | 141 | 50.4% |
| Benign | 40 | 14.3% |
| Pulmonary metastasis | 33 | 11.8% |
| Minimally invasive adenocarcinoma | 17 | 6.1% |
| Squamous cell cancer | 15 | 5.4% |
| Adenocarcinoma in situ | 8 | 2.9% |
| Small cell lung cancer | 6 | 2.1% |
| Other | 19 | 7% |
| Tumor subtype | | |
| Acinar | 61 | 21.8% |
| Papillary | 29 | 10.4% |
| Non-mucinous | 22 | 7.9% |
| Lepidic | 19 | 6.8% |
| Solid | 14 | 5.0% |
| Mucinous | 11 | 3.9% |
| Micropapillary | 5 | 1.8% |
| Keratinizing | 5 | 1.8% |
| Other | 5 | 1.8% |
| Tumor differentiation | | |
| Well-Differentiated | 34 | 17.5% |
| Moderately differentiated | 101 | 52.1% |
| Poorly differentiated | 57 | 29.4% |
| Undifferentiated | 2 | 1.0% |

When examining the utility of in situ TBR measurements, one should consider two other important caveats which include manual region of interest selection and timing. This naturally introduces observer bias where certain areas could be omitted in order to acquire elevated TBR values. Secondly, as mentioned in previous research, there is no standardized way on selecting appropriate variables on imaging processing. This is important as different normal parenchymal surfaces have their intrinsic autofluorescence. Researchers might choose muscle or adipose tissue which have no autofluorescence compared to normal lung surface which has significantly higher background fluorescence. These differences in appropriate variable selections between studies bring inherent bias, lack of generalizability, and lack of standardized reporting which diminishes clinical applicability in real-time surgery. Secondly, timing of in situ TBR calculation is an important consideration for the surgical oncologist. The majority of IMI devices do not calculate TBRs in real time. When TBRs are reported, they are usually calculated on surgical images that have been processed post hoc (FIGS. 8A-8H). Therefore, one needs to critically approach comparison of calculated TBR values obtained during real time fluorescence-guided surgery with results reported in literature.

TABLE 4
Mean TBR values calculated for in situ, ex vivo (back table),
and bisected ex vivo for the entire cohort. Subgroup analysis
of in situ TBR values for various malignancies are also presented.
Adenocarcinoma spectrum lesions, overall, had higher fluorescence
TBRs compared to non- adenocarcinoma lesions

| | Mean | Std. deviation |
|---|---|---|
| In situ TBR | 2.84 | 2.231 |
| Ex vivo TBR | 3.09 | 2.537 |
| Ex vivo TBR bisected tumor | 4.00 | 3.105 |

These results indicate that in a majority of patients who are undergoing oncologic resection with IMI, detection of in situ fluorescence rather than TBR values carries a more important implication as it allows detection of the primary lesion rather than informing on malignant potential. As shown in FIG. 10, NIR tracers can clearly help identify and localize the malignant adenocarcinomas of the lung in situ regardless of the TBR values. NIR dyes allow for excellent localization of lesions and can complement the tactile and visual feedback of the surgeon. Researchers agree that IMI will play an integral role in oncologic surgery in the decades to come and unified fluorescence quantification reporting will need to be agreed upon for it to be generalizable across different subspecialties. Unfortunately, in its current state, in situ TBR measurements fail to fulfill that role.

TABLE 5
Comparison of in situ TBR values for benign and malignant lesions
in the first and third quartiles shows no statistical difference
between similar lesions at different TBR values

| Lesion | In situ TBR <2 | In situ TBR >4.9 | Mann- Whitney- |
|---|---|---|---|
| type | (1st quartile) n | (3rd quartile) n | U Test |
| Benign | 8 | 4 | p = 0.512 |
| Malignant | 22 | 24 | p = 0.145 |

### CONCLUSION

The results of our retrospective analysis show that in situ TBR measurements do not necessarily provide the surgical oncologist with information regarding the malignant potential of the lesion and is not influenced by demographic, technologic, or intrinsic tumor factors. However, as long as the tumor fluoresces and is visualized by the surgeon meaning in situ TBR 91.1, lesions regardless of size can be localized in real time. Surgeons should be careful in interpreting these values for deeper lesions and generalizing it across different types of NIR dyes. Further prospective studies should be performed to elucidate clinical potential of in situ TBR quantification and standardize it across the industry as IMI becomes more ubiquitous in surgical oncology. Although OTL38 is discussed herein as an example, the scope of the present disclosure is not so limited. For example, pafolacianine, NIR fluorochrome, another optical imaging agent, and the like can be used in accordance with certain exemplary embodiments of the present disclosure.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

What is claimed is:

1. A computer-implemented method comprising:
(a) receiving a fluorescence image of a patient tissue;
(b) determining a level of fluorescence for each of a plurality of sections of the fluorescence image;
(c) comparing a level of fluorescence for a section of the fluorescence image to one or more other levels of fluorescence for one or more other sections of the fluorescence image;
(d) determining a tumor-to-background ratio (TBR) value for the level of fluorescence for the section of the fluorescence image based on the comparing, wherein the comparing step is according to the TBR value;
(e) repeating steps (a)-(d) for another fluorescence image after the patient tissue is resected;
(f) receiving a plurality of medical-history parameters of the patient; and
(g) calculating, via a machine-learning model previously trained using at least the TBR values and the medical-history parameters, whether the patient tissue contains a malignant tumor from TBR values of a plurality of sections of the fluorescence image and the plurality of medical-history parameters, and
wherein the machine-learning model is further trained using a location of the patient tissue as a parameter.

2. The computer-implemented method of claim 1, further comprising:
(h) repeating steps (a)-(d) for yet another fluorescence image.

3. The computer-implemented method of claim 1, wherein steps (a)-(d) are performed after resection, but before removal of the patient tissue from the patient's body.

4. The computer-implemented method of claim 1, wherein steps (a)-(d) are performed after resection and before removal of the patient tissue from the patient's body.

5. The computer-implemented method of claim 1, wherein each of the plurality of sections comprise a pixel of the fluorescence image.

6. The computer-implemented method of claim 1, wherein the patient tissue is an organ.

7. The computer-implemented method of claim 6, wherein the organ is a lung.

8. The computer-implemented method of claim 1, wherein the patient tissue contains a tumor or lesion.

9. The computer-implemented method of claim 1, wherein the plurality of medical-history parameters comprise a patient age, a patient gender, a smoking history of the patient, a time period between fluorescence infusion and surgery for the patient, a distance of the patient tissue from a pleural surface, a size of a tumor contained within the patient tissue, a positron emission tomography (PET) standardized uptake value (SUV) for the fluorescence image, an American Society of Anesthesiologists (ASA) classification for the patient, and a length of stay for the patient.

10. The computer-implemented method of claim 1, wherein the machine-learning model comprises an Image Segmentation algorithm.

11. The computer-implemented method of claim 1, further comprising:
categorizing a subset of the plurality of sections of the fluorescence image as background sections of the patient tissue; and
categorizing another subset of the plurality of sections of the fluorescence image as containing sections of tumor of the patient tissue.

12. The computer-implemented method of claim 1, wherein steps (a)-(f) are performed in less than a minute.

13. A method of training the machine learning model of claim 1, comprising:
inputting another fluorescence image of another patient tumor;
inputting plurality of medical-history parameters for the other patient; and
categorizing a subset of a plurality of sections of the other fluorescence image as containing a tumor.

14. The method of claim 13, further comprising:
categorizing whether the tumor is benign or malignant.

15. A smart device configured to implement the method of claim 1, comprising:
an optical imaging system including a camera optic configured to image at least a section of a tissue of a patient, the optical imaging system being configured and adapted to detect near infrared (NIR) fluorescence dye injected into the patient;
a light source; and
a computing device configured and adapted to utilize optical imaging and quantify background and tumor fluorescence in real time based on predictive models.

16. The smart device of claim 15 wherein the computing device is configured and adapted to assess the probability of lesions being malignant.

17. The smart device of claim 15 further comprising processors and memory capable of performing TBR analysis for images captured by the optical imaging system, the TBR analysis including:
delineating areas of high fluorescence;
delineating areas of low fluorescence; and
calculating a TBR from delineating the areas of high fluorescence and the areas of low fluorescence.

18. The smart device of claim 15 wherein the computing device is capable of: detecting fluorescence, determining the TBR, and determining a probability of malignancy.
